(19) United States
(12) Patent Application Publication
(10) Pub. No.: US 2009/0311712 A1
(43) Pub. Date: Dec. 17, 2009

(54) METHOD OF SCREENING MULTIPLE SINGLE NUCLEOTIDE POLYMORPHISMS ASSOCIATED WITH SUSCEPTIBILITY TO SPECIFIC DISEASE OR DRUG RESPONSE

(75) Inventors: Yun-Sun Nam, Seongnam-si (KR); Seung-Hak Choi, Seongnam-si (KR); Jae-Heup Kim, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/543,249

(22) Filed: Aug. 18, 2009

Related U.S. Application Data
(62) Division of application No. 11/454,336, filed on Jun. 16, 2006.

(30) Foreign Application Priority Data
Jun. 16, 2005 (KR) 10-2005-0052042

(51) Int. Cl. C12Q 1/68

(52) U.S. Cl. 435/6

(57) ABSTRACT

Provided is a method of screening multiple single nucleotide polymorphisms (SNPs) having significance with a case group, the method comprising: selecting one or more SNPs from nucleic acid sequences of the case group and a control group; generating all combinable genotype patterns of multiple SNPs comprised of two or more of the selected SNPs; determining frequencies of the genotype patterns from the case group and the control group; and determining and choosing genotype patterns having statistical significance with the case group using the frequencies. According to the method of screening multiple SNPs, multiple SNPs associated with a specific disease or drug can be effectively selected from the entire genome of an individual. Methods of identifying susceptibility of an individual to development of Type II diabetes are also disclosed.

### CROSS-REFERENCE TO RELATED PATENT APPLICATION

This application is a divisional application of U.S. patent application Ser. No. 11/454, 336, filed Jun. 16, 2006, which claims priority to Korean Patent Application No. 10-2005-0052042, filed on Jun. 16, 2005, the disclosure of each of which is incorporated herein in its entirety by reference.

### BACKGROUND OF THE INVENTION

1. Field of the Invention

The present invention relates to a method of screening multi single nucleotide polymorphisms associated with susceptibility to a specific disease or drug.

2. Description of the Related Art

DNA included in human chromosomes instructs cells to make all proteins in the body. The proteins perform vital functions. A polymorphism or mutation that occurs in a DNA sequence that encodes a protein can cause a variation or a mutation in a protein encoded by the DNA and cause abnormal functions in cells. Polymorphisms and mutations in the DNA of individuals are associated with almost all diseases such as infectious disease, cancer and self-immunity disease, even though environmental factors often cause the diseases. Complex interactions among several genes or various polymorphisms or mutations within one gene are known to be the cause of many diseases, as opposed to other diseases caused by only a single polymorphism or mutation in one gene. For example, type 1 and 2 diabetes are known to be associated with multiple genes and each type is associated with a specific pattern of polymorphisms or mutations. On the other hand, cystic fibrosis is known to be able to occur facilitated by one of 300 or more polymorphisms or mutations in one gene.

Additionally, it is known in the field of pharmacogenomics that variations in DNA sequences result in inter-individual differences in reactions to drugs. For example, Evans and Relling (Evans and Relling, Science 286:487-91, 1999), showed that a certain side effect was associated with amino acid mutations in two drug metabolic enzymes, i.e. plasma cholinesterase and glucose-6-phosphate dehydrogenase. By sequencing genes, sequential polymorphisms or mutations in 35 or more drug metabolizing enzymes, 25 or more drug targets and 5 or more drug carriers have been found to be associated with the efficacy or stability of drugs. The obtained data are used to prevent the toxic administration of drugs in hospitals, etc. For example, genetic variations in the thiopurine methyltransferase gene causing a decreased metabolism of 6-mercaptopurine or azathiopurine in patients are usually screened. However, the drug's observed toxicity has not been fully explained by the identified pharmacogenetic marker set. Additionally, the problem that a safe and effective drug for one individual has an insufficient effect or a side effect for another individual is common.

Human genomic sequence polymorphisms are variations in 0.1% of the base sequences in the entire human genome. That is, 99.9% of the human genome in two arbitrarily selected persons are identical while 0.1% are different. Thus, the variations associated with susceptibility to a specific disease or to the effectiveness of or a side effect to a specific drug are less than 0.1% of the human genome. Such polymorphisms include restriction fragment length polymorphisms (RFLPs), short tandem repeats (STRs) and single nucleotide polymorphisms (SNPs). SNPs are variations in single nucleotides among individuals of the same species. When a SNP occurs in a protein coding sequence, the polymorphism may cause the expression of a defective or variant protein. SNPs may also occur in non-coding sequences. Some of these polymorphisms may cause the expression of defective or variant proteins as a result of a defective splicing of mRNA, for example. Other SNPs may have no phenotypic effect.

When SNPs induce a phenotypic expression such as a disease or a reaction to a drug, polynucleotides including the SNPs can be used as a primer or a probe for the diagnosis of the disease and the prediction of the reaction to the drug. Monoclonal antibodies specifically binding with the SNPs can also be used in the diagnosis of the disease and in the prediction of the reaction to the drug. Currently, many research institutes are performing research on the nucleotide sequences and functions of SNPs. The nucleotide sequences and the results of other experiments on identified human SNPs have been put in databases for easy access. Even though a great many SNPs in human genome or cDNA have been found, the phenotype effects of most SNPs have not been completely revealed. The functions of most SNPs have not been found.

Various methods of screening SNPs have been used. Known methods involve the selection of a specific region in the genome that is known to be associated with a disease or with drug response and the order of the incidence rate or the presence of the disease or drug response with regard to the possible genotype patterns of the selected region. However, the prognosis of the disease and the prediction of the reaction to the drug are not available when only one SNP or a set of SNPs in a specific region is considered.

The present inventors found a method of screening genotype patterns of a multiple SNP including two or more SNPs associated with susceptibility to a disease or to effectiveness of a drug selected from entire nucleic acid sequences of individuals.

### SUMMARY OF THE INVENTION

The present invention provides a method of screening multiple single nucleotide polymorphisms (SNPs) associated with susceptibility to a specific disease or with drug response from the entire nucleic acid sequence of an individual.

According to an aspect of the present invention, there is provided a method of screening multiple SNPs having significance with a case group. The method includes selecting one or more SNPs from nucleic acid sequences of the case group and a control group, generating all combinable genotype patterns of multiple SNPs composed of two or more of the selected SNPs, determining frequencies of the genotype patterns from the case group and the control group, and determining and choosing genotype patterns having statistical significance with the case group using the frequencies.

The method may include isolating substantially identical nucleic acids from a plurality of individuals of the case group and the control group in advance of the selecting one or more SNPs from nucleic acid sequences of the case group and the control group.

Also disclosed herein are methods of identifying susceptibility of an individual to development of Type II diabetes. In an embodiment, the method comprises determining the genotype of the individual at the SNPS of a multiple SNP locus shown in Table 5 and identifying the individual as at risk of developing Type II diabetes if the determined genotypes of the individual at the SNPs of the selected multiple SNP locus match the genotypes shown in Table 5. In another embodiment, the method comprises determining the presence or absence in the individual of a risk factor allele at a SNP shown in Table 3 and identifying the individual as at risk of developing Type II diabetes if the risk factor allele of the selected SNP is present in the individual.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart of a method of screening a multiple SNP according to an embodiment of the present invention; and

FIG. 2 illustrates a concept of a method of screening a multiple SNP according to another embodiment of the present invention.

### DETAILED DESCRIPTION OF THE INVENTION

Hereinafter, the present invention will be described more fully with reference to the accompanying drawings, in which exemplary embodiments of the invention are shown.

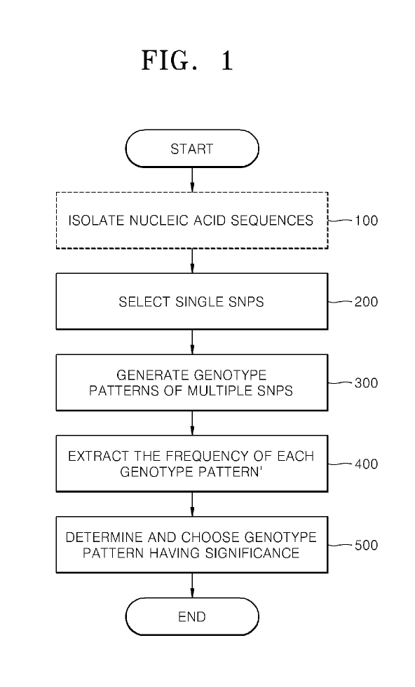

FIG. 1 is a flowchart of a method of screening a multiple SNP according to an embodiment of the present invention. In FIG. 1, the dotted line indicates an optional stage.

Referring to FIG. 1, the method of screening a multiple SNP includes selecting SNPs (operation 200); generating genotype patterns of multiple SNPs (operation 300); determining frequencies (operation 400); and determining and choosing genotype patterns having significance (operation 500). In the present embodiment, the method further includes isolating nucleic acid sequences (operation 100).

The stages of the method of screening multiple SNPs according to an embodiment of the present invention will now be described in greater detail.

Isolating Nucleic Acid Sequences

Substantially identical nucleic acid sequences are isolated from a plurality of individuals of a case group and a control group (operation 100).

When isolated nucleic acid sequences are already prepared, the nucleic acid sequences can be used without the operation 100 of isolating nucleic acid sequences.

However, when nucleic acid sequences are not prepared, substantially identical nucleic acid sequences can be isolated from a plurality of individuals of the case group and the control group in operation 100.

In an embodiment of the present invention, the case group is a group showing abnormal phenotypic expressions and the control group is a group not showing abnormal phenotypic expressions.

Particularly, the case group may have a susceptibility to a specific disease and the control group may not have a susceptibility to the disease. The members of the group having the susceptibility to the specific disease may already have been diagnosed with the disease. In the detailed description, the term “disease” is often used to indicate a disordered condition, trait or characteristic in an organic body, but is not limited thereto. For example, the disordered condition, trait or characteristic may occur physically, physiologically or psychologically, and may or may not have any symptoms.

Alternatively, the case group may not have susceptibility to a certain drug and the control group may have susceptibility to the drug. Herein, the susceptibility to a drug indicates susceptibility to the effect of the drug. Alternatively, the case group may have susceptibility to a side effect of a drug and the control group may not have susceptibility to the side effect of the drug.

Herein, an individual may be a specific single organism such as an animal, a parasite living in a human, or a bacterium, for example, a human.

Herein, substantially identical nucleic acid may have at least 80% identical sequences, for example, at least 85% identical sequences, or at least 95% identical sequences. The degree of nucleic acid sequence identity may depend on the host of the nucleic acids. For example, in a comparison among members of the same species, at least 95% of sequences may be identical.

Particularly, the nucleic acid sequence may be exon, or exon and intron, for example, intron, exon and sequences between genes. The nucleic acid sequence may be partial sequences obtained from entire sequences of an individual, or may be entire sequences of an individual. Repeated regions in the nucleic acid sequences known to be completely identical in all members of the same species may be removed in the experiments for economic purposes.

Nucleic acid sequences may be isolated using one of the methods known to those skilled in the art. For example, to obtain pure nucleic acid, after contents of a cell are extracted, differential precipitation, column chromatography, an extracting method using an organic solvent, etc. may be carried out. The extract of the cell contents may be prepared using a standard technique such as chemical or mechanical dissolution of cells. The extract may be filtered, centrifuged and/or treated with a chaotropic salt such as guanidium isothiocynate, urea, or an organic solvent such as phenyl and/or HCCl₃ to prevent contamination and remove interfering proteins. When chaotropic salt is used, the salt may be removed from the sample including nucleic acid. The removal of the salt can be carried out using a standard technique such as sedimentation, filtering or size exclusion chromatography.

The nucleic acid may be amplified before determining the existence of polymorphisms in the nucleic acid. An amplification technique known in the art can be used, and may include, but is not limited to, a PCR. The PCR may be carried out using a material and method known in the art.

A PCR may be performed to amplify the entire nucleic acid sequences of an individual or may be performed to amplify partial nucleic acid sequences around a SNP published in a known database.

Selecting Single SNPs

One or more SNPs are selected from nucleic acid sequences of each of the case group and the control group (operation 200).

The nucleic acid, more particularly the nucleotides around SNPs, is sequenced to select SNPs. DNA sequencing can be carried out using a conventional method known to those skilled in the art.

DNA sequencing methods have been introduced by Sambrook et al., (Molecular Cloning, New York, 1989) and Ausubel et al., (Current Protocols in Molecular Biology, New York, 1997). The methods can be used for determining the same regions of DNA sequences where a noticeable variation exists when comparing the sequences.

The DNA may be sequenced using a known automatic sequencing device, for example, Hamilton Micro Lab 2200 (Hamilton, Reno), Peltier Thermal Cycler (PTC200; MJ Research, Watertown), ABI Catalyst and 373 or 377 DNA Sequencer (Perkin Elmer, Wellesley).

Sequencing may also be carried out using a commercially available capillary electrophoresis system. In the capillary electrophoresis system, electrophoresis separation, a laser activated four difference color fluorescent dying, and a floating polymer for detecting the wavelength of light emitted by an electric charge can be used.

A hybridization technique such as the use of DNA chips (oligonucleotide array) may be used for sequencing. The details on the usage of a DNA chip for detecting SNPs were introduced by Lipshultz et al. (U.S. Pat. No. 6,300,063) and Chee et al. (U.S. Pat. No. 5,837,832).

In an embodiment of the present invention, Matrix Assisted Laser Desorption and Ionization-Time of Flight Mass Spectrometry (MALDI-TOF MS) can be used for the sequencing.

The MALDI-TOF MS is a method of ionizing a biopolymer by irradiating a pulse laser onto the biopolymer mixed with matrix molecules. When a matrix molecule such as a 3-hydroxypicolinic acid and a material to be analyzed is exposed to a laser beam, the matrix molecule absorbs the laser beam, transfers energy and protons to the material, and ionizes the material. The material exposed to the laser beam flies with the ionized matrix in a vacuum to a detector. The flying time to the detector is calculated to determine the mass. A light material reaches the detector in shorter amount of time than a heavy material. The SNP sequences in a target DNA may be determined based on differences in mass and known SNP sequences.

After analyzing the SNP sequences, it is determined whether reported SNPs are actual polymorphic sites. In fact, sometimes a reported SNP proves not to be a real polymorphic site after analysis in a particular population.

In selecting SNPs, SNPs satisfying the Hardy-Weinberg Equilibrium Law may be selected from the control group.

The nucleic acid sequences of a successful SNP selection should have values in predetermined ranges as follows.

For example, when the sequencing is performed using a plate having multiple wells, a call rate of the nucleic acid may be 95% or greater, an IRF value of the nucleic acid may be 5% or less and a blank well of the nucleic acid may be 5% or less. The call rate indicates the ratio of the number of samples successfully measured to the number of total samples used for an experiment. If the call rate is less than 95%, the sample should be thrown away and the experiment should be restarted. When some of the samples used in the experiments are tested twice, the IRF indicates the percentage of the sample in which the two data are not identical. When the IRF value is higher than 5%, the entire sample should be thrown away. Blank well indicates a proportion of detected signals to the total case that is a control group in which the experiments are performed with only water. When the blank well is higher than 5%, the entire sample should be thrown away.

Generating Genotype Patterns of Multiple SNPs

All combinable genotype patterns of multiple SNPs composed of two or more of the selected SNPs are generated (operation 300).

First, the multiple SNPs are generated by selecting two or more SNPs.

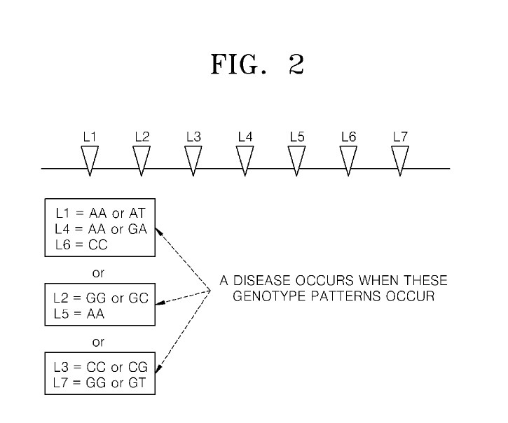

FIG. 2 conceptually illustrates a part of a method of screening multiple SNPs according to another embodiment of the present invention.

In FIG. 2, 7 SNPs are illustrated. A small number of SNPs are illustrated for better understanding. A multiple SNP, i.e. a combination of at least two SNPs among the 7 SNPs, is generated. The number of possible multiple SNPs composed of k SNPs selected from n SNPs is represented by ₙCₖ. When two SNPs are selected from the 7 SNPs, the number of possible multiple SNPs is ₇C₂; when three SNPs are selected, the number of possible multiple SNPs is ₇C₃; when four SNPs are selected, the number of possible multiple SNPs is ₇C₄; when five SNPs are selected, the number of possible multiple SNPs is ₇C₅; when six SNPs are selected, the number of possible multiple SNPs is ₇C₆; and when seven SNPs are selected, the number of possible multiple SNPs is ₇C₇. Therefore, the number of possible multiple SNPs selected from n SNPs can be calculated using formula 1 below:

$${\sum }_{k = 2}^{n} {}_{n}^{}{C}{}_{k}^{} , \qquad ( 1 )$$

where, n=the number of SNPs. Thus, the total number of possible multiple SNPs which can be derived from the 7 single SNPs is 110.

Next, all combinable genotype patterns of the multiple SNPs are generated. When the two alleles occurring at a SNP are A1/A2, the genotype pattern of the SNP site may be one of the following: A1A1, A1A2 and A2A2. Furthermore, one of the following five genotype groupings for the SNP may be included in the predictive genotype pattern for the multiple SNP: A1A1, A1A2, A2A2, A1A1 or A1A2, and A1A2 or A2A2. For example, if the genotype of a single SNP significantly associated with the diseased case group is A1A1 or A1A2, A1 can be determined to be a risk factor and if the genotype of a single SNP significantly associated with the diseased case group is A1A2 or A2A2, A2 can be determined to be a risk factor. That is, when the multiple SNP includes one of the five genotype groupings for each SNP, a possible number of combinable genotype patterns of the multiple SNP composed of k single SNPs is 5^{k}. Therefore, the possible number of combinable genotype patterns of the multiple SNP that is composed of the two or more SNPs can be calculated using formula 2 below:

$${\sum }_{k = 2}^{n} {}_{n}^{}{C}{}_{k}^{} \cdot {5}^{k} , \qquad ( 2 )$$

where, n=the number of SNPs.

According to FIG. 2, the number of possible combinable genotype patterns of the multiple SNP which is comprised of the two or more SNPs selected from 7 SNPs is

₇C₂·5²+₇C₃·5³+₇C₄·5⁴+₇C₅·5⁵+₇C₆·5⁶+₇C₇·5⁷=279,900.

Determining Frequencies

The frequencies of the genotype patterns of the case group and the control group are determined (operation 400).

That is, the numbers of individuals in the case group having and not having a certain genotype pattern are respectively calculated. In the same way, the numbers of individuals in the control group having and not having the genotype pattern are respectively calculated.

A contingency table may be prepared using the determined frequencies. The contingency table may be Table 1 below.

TABLE 1

| | Having the genotype | Not having the | |
|---|---|---|---|
| | pattern | genotype pattern | Total |
| The case group | a | b | a + b |
| The control group | c | d | c + d |
| Total | a + c | b + d | a + b + c + d |

Determining and Choosing Genotype Patterns Having Significance

The genotype patterns having a statistical significance to the case group are determined and chosen using the determined frequencies (operation 500).

Various statistical significance tests can be used. Multiple SNPs and the genotype patterns thereof representing a high significance can be determined using all of the various significance tests.

The statistical significance can be determined in consideration of genotype pattern ratio and genotype pattern difference. The genotype pattern ratio and the genotype pattern difference are calculated using the equations indicated below.

Genotype pattern ratio=(number of individuals in the case group having a certain genotype pattern)/(number of individuals in the control group having the genotype pattern)

Genotype pattern difference=(number of individuals in the case group having a certain genotype pattern)−(number of individuals in the control group having the genotype pattern)

For example, based on the information in Table 1, the genotype pattern ratio and the genotype pattern difference may be represented as follows:

Genotype pattern ratio=a/c.

Genotype pattern difference=a−c.

Genotype patterns having greater genotype pattern ratios and greater genotype pattern difference have a high statistical significance to the case group. For example, when the genotype pattern ratio is 2 or more and the genotype pattern difference is 0.1×(total number of individuals in the case group) or higher (in Table 1, the genotype pattern difference is 0.1×(a+b) or higher)), the genotype pattern is determined to have high statistical significance to the case group.

The statistical significance can be determined using additional significant tests such as an odds ratio, a 95% confidence interval and a 99% confidence interval of the odds ratio.

The odds ratio indicates the ratio of the probability of the genotype patterns of the multiple SNP being in the case group to the probability of the genotype patterns of the multiple SNP being in the control group. For example, using the data in Table 1, the odds ratio may be represented as follows:

Odds ratio=ad/bc.

If the odds ratio exceeds 1, there is significance between the genotype pattern of the multiple SNP and the case group. The degree of the significance increases with the odds ratio. Significance may be determined when the odds ratio is 2 or greater, for example, 3 or greater.

95% and 99% confidence intervals are regions in which 95% and 99% of the odds ratio are distributed respectively, and are obtained using the below formulas. When 1 is within the confidence interval, i.e. the lower bound is below 1 and the upper bound is above 1, it is estimated that there is no association between the multiple SNP and the disease.

$$\begin{matrix}95 \% confidence interval = ( lower bound , upper bound ) \\ = ( \begin{matrix}odds ratio \times exp ( - 1.960 \sqrt{V} ) , \\ odds ratio \times exp ( 1.960 \sqrt{V} )\end{matrix} ) ,\end{matrix}$$

$$where$$

$$V = 1 / a + 1 / b + 1 / c + 1 / d . \\ \begin{matrix}99 \% confidence interval = ( lower bound , upper bound ) \\ = ( \begin{matrix}odds ratio \times exp ( - 2.576 \sqrt{V} ) , \\ odds ratio \times exp ( 2.576 \sqrt{V} )\end{matrix} ) ,\end{matrix}$$

$$where$$

$$V = 1 / a + 1 / b + 1 / c + 1 / d .$$

Significance may be determined when the lower bound of the confidence interval is 2 or greater, for example 3 or greater.

The statistical significance may be determined in another way, for example, by using the p-value of Fisher's exact test.

Fisher's exact test may be carried out using a known method to obtain the p-value (Fisher, R. A., The logic of inductive inference, Journal of the Royal Statistical Society Series A, 1935. 98: p. 39-54).

When the p-value is 0.05 or less, the genotype patterns may be regarded as statistically significant.

The statistical significance, p-value may be corrected by multiple testing method.

Multiple testing methods are known to those skilled in the art. For example, a multiple testing method may be Bonferroni correction with discrete distributions (Westfall, P. H. A. W., R. D., Multiple tests with discrete distributions. The American Statistician, 1997. 51: p. 3-8); a step-down method (Westfall, E. A., Multiple Comparisons and Multiple Tests: Using the Sas System. 1999: SAS Institute); a step-up method (Westfall, E. A., Multiple Comparisons and Multiple Tests: Using the Sas System. 1999: SAS Institute); permutation method (Westfall, E. A., Resampling-based multiple testing: Examples and methods for p-value adjustment. 1993: Wiley); or Bootstrap method (Westfall, E. A., Resampling-based multiple testing: Examples and methods for p-value adjustment. 1993: Wiley). The p-value can be corrected using one of the listed methods.

The multiple SNPs and the genotype patterns thereof satisfying at least one of the tests, preferably all the tests, are determined to have the statistical significance to the case group.

Also disclosed herein are methods of identifying susceptibility of an individual to development of Type II diabetes.

In an embodiment, the method comprises determining the genotype of the individual at the SNPS of a multiple SNP locus shown in Table 5 and identifying the individual as at risk of developing Type II diabetes if the determined genotype pattern of the individual at the SNPs of the selected multiple SNP locus match the genotype pattern shown in Table 5.

In another embodiment, the method comprises determining the presence or absence in the individual of a risk factor allele at a SNP shown in Table 3 and identifying the individual as at risk of developing Type II diabetes if the risk factor allele of the selected SNP is present in the individual.

The present invention will now be described in greater detail with reference to the following examples. The following examples are for illustrative purposes only and are not intended to limit the scope of the invention.

### Example 1

### Selecting Multiple SNPs Associated with Type 2 Diabetes

It is known that 90 to 95% of all patients having diabetes have type 2 diabetes. In the present Example of the present invention, multiple SNPs associated with type 2 diabetes mellitus (DM2) were selected using a method of screening according to an embodiment of the present invention. Type 2 diabetes tends to develop in people who have an abnormal amount of insulin or have low sensitivity to insulin. Patients having type 2 diabetes have a wide range of sugar levels in their blood.

DNA was isolated from the blood of individuals of a case group diagnosed with type 2 diabetes and treated, and DNA was isolated from a control group not having symptoms of type 2 diabetes, each group consisting of Koreans, and then an appearance frequency of a specific SNP was analyzed. The SNPs of the Examples were selected from either a public database (NCBI dbSNP:) or a commercial database available from Sequenom. The SNPs were analyzed using a primer close to the selected SNPs.

1-1. Preparation of DNA Sample

DNA was extracted from blood of the case group consisting of 300 patients diagnosed with type 2 diabetes and treated, and DNA was extracted from the control group consisting of 300 normal persons not having symptoms of type 2 diabetes. Chromosomal DNA extraction was carried out using a known molecular cloning extraction method (A Laboratory Manual, p 392, Sambrook, Fritsch and Maniatis, 2nd edition, Cold Spring Harbor Press, 1989) and guidelines of a commercially available kit (Gentra system, D-50K). Only DNA having a purity of at least 1.7, measured using UV light (260/280 nm), was selected from the extracted DNA and used.

1-2. Amplification of Target DNA

The target DNA having a certain DNA region including a SNP to be analyzed was amplified using a PCR. The PCR was performed using a general method and the conditions were as indicated below. 2.5 ng/ml of the chromosomal DNA was prepared and then the following PCR reaction solution was prepared.

| | |
|---|---|
| Water (HPLC grade) | 2.24 μl |
| 10 × buffer (containing 15 mM MgCl₂, 25 mM MgCl₂) | 0.5 μl |
| dNTP mix (GIBCO) (25 mM/each) | 0.04 μl |
| Taq pol (HotStart) (5 U/μl) | 0.02 μl |
| Forward/reverse primer mix (1 μM/each) | 0.02 μl |
| DNA | 1.00 μl |
| Total volume | 5.00 μl |

The forward and reverse primers were selected upstream and downstream from the SNPs at a proper position using a known database. Several primers are listed in Table 2.

Thermal cycling of PCR was performed by maintaining the temperature at 95° C. for 15 minutes, cycling the temperature from 95° C. for 30 seconds, to 56° C. for 30 seconds, to 72° C. for 1 minute a total 45 times, maintaining the temperature at 72° C. for 3 minutes and then stored at 4° C. As a result, target DNA fragments containing 200 nucleotides or less were obtained.

1-3. Selection of SNP

SNP analysis of the target DNA fragments was performed using a homogeneous Mass Extend (hME) technique established by Sequenom. The principle of the hME technique is as follows. First, a primer, also called an extension primer, complementary to bases up to just before the SNP of the target DNA fragment was prepared. Next, the primer was hybridized with the target DNA fragment and DNA polymerization was facilitated. At this time, added to the reaction solution was a reagent (Termination mix; e.g. ddTTP) for terminating the polymerization after the complementary base was added to a first allele (e.g. ‘A’ allele) among the subject SNP alleles. As a result, when the target DNA fragment included the first allele (e.g. ‘A’ allele), a product having only one base complementary to the first allele (e.g. ‘T’) added was obtained. On the other hand, when the target DNA fragment included a second allele (e.g. ‘G’ allele), a product having a base complementary to the second allele (e.g. ‘C’) and extending to the first allele base (e.g. ‘A’) was obtained. The length of the product extending from the primer was determined using mass analysis to determine the type of allele in the target DNA. Specific experimental conditions were as follows.

First, free dNTPs were removed from the PCR product. To this end, 1.53 μl of pure water, 0.17 μl of an hME buffer and 0.30 μl of shrimp alkaline phosphatase (SAP) were added to a 1.5 ml tube and mixed to prepare a SAP enzyme solution. The tube was centrifuged at 5,000 rpm for 10 seconds. Then, the PCR product was put into the SAP solution tube, sealed, maintained at 37° C. for 20 minutes and at 86° C. for 5 minutes, and then stored at 4° C.

Next, a homogenous extension was performed using the target DNA product as a template. The reaction solution was as indicated below.

| | |
|---|---|
| Water (nanopure grade) | 1.728 μl |
| hME extension mix (10 × buffer containing 2.25 mM d/ddNTPs) | 0.200 μl |
| Extension primer (each 100 μM) | 0.054 μl |
| Thermosequenase (32 U/μl) | 0.018 μl |
| Total volume | 2.00 μl |

The reaction solution was mixed well and spin down centrifuged. A tube or plate containing the reaction solution was sealed, maintained at 94° C. for 2 minutes, cycled from 94° C. for 5 seconds, to 52° C. for 5 seconds, to 72° C. for 5 seconds a total of 40 times, and then stored at 4° C. The obtained homogeneous extension product was washed with a resin (SpectroCLEAN, Sequenom, #10053) and salt was removed. Several of the primers used for the homogeneous extension are disclosed in Table 2.

TABLE 2

| | Primer for target | | |
|---|---|---|---|
| | DNA amplification | | |
| | (SEQ ID NO:) | | Extension primer |
| Name of Marker | Forward primer | Reverse primer | (SEQ ID NO:) |
| DMX_009 | 13 | 14 | 15 |
| DMX_011 | 16 | 17 | 18 |
| DMX_029 | 19 | 20 | 21 |
| DMX_032 | 22 | 23 | 24 |
| DMX_033 | 25 | 26 | 27 |
| DMX_044 | 28 | 29 | 30 |
| DMX_056 | 31 | 32 | 33 |
| DMX_104 | 34 | 35 | 36 |
| DMX_154 | 37 | 38 | 39 |
| DMX_058 | 40 | 41 | 42 |
| DMX_101 | 43 | 44 | 45 |
| DMX_131 | 46 | 47 | 48 |

A mass analysis was performed on the obtained extension product to determine the sequence of a polymorphic site using MALDI-TOF MS.

Only sites polymorphic in the study population were selected using the results of sequencing SNPs of the target DNA through MALDI-TOF MS. In addition, SNPs were selected for which the genetic makeup of the alleles had a constant frequency in the control group according to Mendel's Law of inheritance and the Hardy-Weinberg Law. The experiments were regarded as successful when the call rate was 95% or greater, the IRF value was 5% or less and the blank well was 5% or greater.

87 SNP sites were selected as a result of the series of selections. Several of the SNPs are indicated in Tables 3 and 4. Each allele may exist in the form of a homozygote or a heterozygote in an individual.

TABLE 3

| | SEQ | | | | | | | | | | | |
|---|---|---|---|---|---|---|---|---|---|---|---|---|
| | ID | Alleles | | Allele frequency | | | Genotype frequency | | | | | |
| ASSAY_ID | NO: | A1 | A2 | cas_A2 | con_A2 | Delta | cas_A1A1 | cas_A1A2 | cas_A2A2 | con_A1A1 | con_A1A2 | con_A2A2 |
| DMX_009 | 1 | T | G | 0.664 | 0.737 | 0.073 | 31 | 138 | 129 | 19 | 119 | 161 |
| DMX_011 | 2 | A | G | 0.866 | 0.931 | 0.065 | 7 | 66 | 225 | 1 | 39 | 258 |
| DMX_029 | 3 | C | A | 0.057 | 0.104 | 0.047 | 268 | 28 | 3 | 241 | 52 | 5 |
| DMX_032 | 4 | T | A | 0.718 | 0.593 | 0.125 | 26 | 117 | 157 | 51 | 142 | 107 |
| DMX_033 | 5 | T | C | 0.816 | 0.9 | 0.084 | 10 | 89 | 198 | 4 | 51 | 239 |
| DMX_044 | 6 | A | T | 0.846 | 0.787 | 0.059 | 7 | 78 | 213 | 15 | 93 | 181 |
| DMX_056 | 7 | A | G | 0.362 | 0.273 | 0.089 | 123 | 137 | 40 | 160 | 116 | 24 |
| DMX_104 | 8 | T | C | 0.274 | 0.204 | 0.07 | 158 | 115 | 24 | 184 | 95 | 12 |
| DMX_154 | 9 | A | G | 0.269 | 0.199 | 0.07 | 153 | 131 | 15 | 187 | 100 | 9 |
| DMX_058 | 10 | A | G | 0.315 | 0.382 | 0.067 | 138 | 131 | 28 | 111 | 144 | 41 |
| DMX_101 | 11 | A | T | 0.38 | 0.316 | 0.064 | 118 | 136 | 46 | 138 | 133 | 28 |
| DMX_131 | 12 | A | T | 0.441 | 0.376 | 0.065 | 97 | 139 | 62 | 118 | 136 | 44 |

| | association: chi-square | | Odds ratio (multiple ratio) | | | | | | call rate of | |
|---|---|---|---|---|---|---|---|---|---|---|
| | (df = 2) | | Risk | | | | HWE | | sample | |
| ASSAY_ID | Chi_value | Chi_exact_pValue | factor | | OR | CI | con_HW | cas_HW | cas_call_rate | con_call_rate |
| DMX_009 | 7.814 | 0.0201002 | A1 | T | 1.42 | (1.106, 1.82) | .195, HWE | .424, HWE | 0.99 | 1 |
| DMX_011 | 13.698 | 0.0010608 | A1 | A | 2.1 | (1.414, 3.115) | .026, HWE | .948, HWE | 0.99 | 0.99 |
| DMX_029 | 9.131 | 0.0104069 | A1 | C | 1.93 | (1.247, 2.975) | 1.514, HWE | 13.034, HWD | 1 | 0.99 |
| DMX_032 | 20 | 0.00004.541 | A2 | A | 0.57 | (0.449, 0.728) | .148, HWE | .582, HWE | 1 | 1 |
| DMX_033 | 16.718 | 0.0002343 | A1 | T | 2.02 | (1.434, 2.831) | 2.023, HWE | .005, HWE | 0.99 | 0.98 |
| DMX_044 | 6.687 | 0.0353052 | A2 | C | 0.68 | (0.501, 0.91) | .452, HWE | .013, HWE | 0.99 | 0.96 |
| DMX_056 | 10.581 | 0.0050404 | A2 | T | 0.66 | (0.52, 0.848) | .283, HWE | .041, HWE | 1 | 1 |
| DMX_104 | 7.821 | 0.0200309 | A2 | G | 0.68 | (0.519, 0.891) | .011, HWE | .284, HWE | 0.99 | 0.97 |
| DMX_154 | 9.045 | 0.0108603 | A2 | C | 0.68 | (0.515, 0.886) | .768, HWE | 3.616, HWE | 1 | 0.99 |
| DMX_058 | 5.99 | 0.0500401 | A1 | A | 1.34 | (1.057, 1.708) | 0.308, HWE | 0.112, HWE | 0.99 | 0.99 |
| DMX_101 | 5.973 | 0.0504718 | A2 | T | 0.75 | (0.594, 0.957) | 0.166, HWE | 0.465, HWE | 1 | 1 |
| DMX_131 | 5.14 | 0.0765166 | A2 | T | 0.76 | (0.605, 0.961) | 0.194, HWE | 0.946, HWE | 0.99 | 0.99 |

TABLE 4

| | | Alleles | | No. of | | | | | SNP | Amino acid |
|---|---|---|---|---|---|---|---|---|---|---|
| ASSAY_ID | rs number | A1 | A2 | chromosome | Location | Band | Gene | Explanation | function | change |
| DMX_009 | rs1394720 | T | G | 11 | 4533242 | 11p15.4 | intergenic | n | intergenic | no change |
| DMX_011 | rs488115 | A | G | 11 | 74409538 | 11q13.4 | intergenic | n | intergenic | no change |
| DMX_029 | rs2051672 | C | A | 17 | 5847149 | 17p13.2 | intergenic | n | intergenic | no change |
| DMX_032 | rs1943317 | T | A | 18 | 62419479 | 18q22.1 | intergenic | n | intergenic | no change |
| DMX_033 | rs929476 | T | C | 19 | 33499519 | 19q12 | intergenic | n | intergenic | no change |
| DMX_044 | rs1984388 | A | T | 22 | 30658575 | 22q12.3 | intergenic | n | intergenic | no change |
| DMX_056 | rs752139 | A | G | 5 | 176000000 | 5q35.2 | PC-LKC | protocadherin | intron | no change |
| | | | | | | | | LKC | | |
| DMX_104 | rs492220 | T | C | 1 | 94254590 | 1p22.1 | ABCA4 | ATP45; binding | intron | no change |
| | | | | | | | | cassette, | | |
| | | | | | | | | sub45; family A | | |
| | | | | | | | | (ABC1), | | |
| | | | | | | | | member 4 | | |
| DMX_154 | rs197367 | A | G | 7 | 36219096 | 7p14.2 | ANLN | anillin, actin | coding-nonsynon | K->R |
| | | | | | | | | binding protein | | |
| | | | | | | | | (scraps | | |
| | | | | | | | | homolog, | | |
| | | | | | | | | Drosophila) | | |
| DMX_058 | rs1340266 | A | G | 6 | 102000000 | 6q16.3 | GRIK2: | glutamate | Intron: no | no change |
| | | | | | | | GRIK2 | receptor, | info | |
| | | | | | | | | ionotropic, | | |
| | | | | | | | | kainate 2 | | |
| DMX_101 | rs1316909 | A | T | 1 | 157000000 | 1q23.2 | 0 | n | 0 | 0 |
| DMX_131 | rs1377188 | A | T | 18 | 29732602 | 18q12.1 | NOL4: | nucleolar | Intron: no | no change |
| | | | | | | | NOL4 | protein 4 | info | |

Here, ‘Assay_ID’ indicates the name of a SNP.

‘Alleles’ are the bases observed at a particular polymorphic site. Here, ‘A1’ and ‘A2’ respectively represent the low mass allele and the high mass allele in sequencing experiments using the hME technique (Sequenom), and are arbitrarily designated for convenience of experiments.

SEQ ID NO is the sequence identification number including the SNP in which the polymorphism is positioned at the 101^{st} nucleotide.

‘allele frequency’ is the frequency at which the alleles occur. ‘cas_A2’, ‘con_A2’ and ‘Delta’ respectively indicate the frequency of allele ‘A2’ in the case group, the frequency of allele ‘A2’ in the control group and the absolute value of the difference between ‘cas_A2’ and ‘con_A2’. ‘cas_A2’ is given by (the frequency of the genotype ‘A2A2’×2+the frequency of the genotype ‘A1A2’)/(the number of samples of the case group×2) and ‘con_A2’ is given by (the frequency of the genotype ‘A2A2’×2+the frequency of the genotype ‘A1A2’)/(the number of samples of the control group×2).

‘Genotype frequency’ indicates the frequency of each genotype. ‘Cas_A1A1, cas_A1A2, cas_A2A2, con_A1A1, con_A1A2 and con_A2A2 respectively indicate the number of individuals having the genotypes A1A1, A1A2 and A2A2 in the case group and A1A1, A1A2 and A2A2 in the control group.

‘Chi-square (df=2)’ indicates a chi-square value when the degree of freedom is 2. ‘Chi-value’ is obtained through the chi-square test and is used for p-value calculation. ‘Chi-exact-p-value’ indicates the p-value of Fisher's exact test of chi-square test, and is a variable used for inspecting more accurate statistical significance since the chi-square test results may be inaccurate when the number of genotypes is less than 5. When the p-value was 0.05 or less, it was judged that the genotype between the case group and the control group was not identical, i.e., significant.

‘HWE’ indicates the condition of Hardy-Weinberg Equilibrium. ‘Con_HWX’ and ‘cas_HWE’ respectively indicates the Hardy-Weinberg Equilibrium in the control group and the case group.

A chi-value of 6.63 or higher (p-value=0.01, df=1) is regarded as Hardy Weinberg Disequilibrium (HWD) and a chi-value of less than 6.63 is regarded as Hardy Weinberg Equilibrium (HWE).

‘Call rate’ indicates the ratio of the number of samples having successful results to the total number samples used in the experiments. ‘Cas_call_rate’ and ‘con_call_rate’ are respectively the ratios of successfully analyzed ratios of genotypes used for the case group and the control group to the total number of samples in each group.

1-4. Generating Genotype Patterns of Multiple SNP and Determining the Frequency

All combinable genotype patterns of the multiple SNPs were generated. The multiple SNPs consisted of 2 to 4 SNPs selected from the 87 SNPs of the case group consisting of 300 patients having type 2 diabetes and the control group consisting of 300 normal persons.

The number of genotype patterns of multiple SNPs consisting of 2 SNPs was 93,525. The number of genotype patterns of multiple SNPs consisting of 3 SNPs was 13,249,375. The number of genotype patterns of multiple SNPs consisting of 4 SNPs was 1,391,184,375.

The frequencies of the genotype patterns of the multiple SNPs were determined from the case group and the control group. A contingency table similar to Table 1 was prepared using the determined frequencies.

1-5. Determining and Choosing Genotype Patterns Having Statistical Significance

The genotype patterns having significance to the case group were determined using the frequencies of genotype patterns of the multiple SNPs in the case group and the control group.

In a first screening, multiple SNPs having a genotype pattern ratio of 2 or greater and a genotype pattern difference of 30 or greater were selected. Among the selected multiple SNPs, multiple SNPs having a genotype pattern ratio of 3 or greater and a genotype pattern difference of 35 or greater were selected for more significant multiple SNP selection,

In a second screening, genotype patterns of multiple SNPs having an odds ratio of 3 or greater, a 95% confidence interval with a lower bound of 2 or greater and a 99% confidence interval with a lower bound of 2 or greater were selected. When the odds ratios and the lower bounds of the 95% and 99% confidence intervals exceed 1.0, the results are statistically significant. However, the required standards were respectively set to 3, 2 and 2 in order to select the most effective markers.

In a third screening, genotype patterns of multiple SNPs having a p-value of Fisher's exact test of 0.05 or less were selected.

In a fourth screening, the p-value was corrected using Bonferroni correction with discrete distributions.

Several genotype patterns that were determined and chosen are listed in Table 5.

TABLE 5

| | | Frequency | Frequency | | | | |
|---|---|---|---|---|---|---|---|
| | | of the | of the | | 95% | | Bonferroni |
| | | case | control | Odds | confidence | | adjusted |
| No. | Genotype Pattern | group | group | ratio | interval | Fisher p-value | p-value |
| 1 | DMX_011 = AA or AG | 59 | 19 | 3.62 | (2.1, 6.24) | 0.0000014 | 0.0508 |
| | DMX_044 = TT | | | | | | |
| 2 | DMX_029 = CC | 94 | 31 | 3.96 | (2.54, 6.18) | 0.000000000225 | 0.000532 |
| | DMX_032 = AA | | | | | | |
| | DMX_056 = AG or GG | | | | | | |
| 3 | DMX_032 = TA or AA | 70 | 23 | 3.67 | (2.22, 6.06) | 0.000000126 | 0.362 |
| | DMX_033 = TT or TC | | | | | | |
| | DMX_131 = AT or TT | | | | | | |
| 5 | DMX_009 = TT or TG | 62 | 17 | 4.34 | (2.47, 7.62) | 0.0000000522 | 0.143 |
| | DMX_101 = AT or TT | | | | | | |
| | DMX_154 = AG or GG | | | | | | |
| 6 | DMX_029 = CC | 71 | 23 | 3.73 | (2.26, 6.17) | 0.0000000752 | 0.22 |
| | DMX_058 = AA | | | | | | |
| | DMX_104 = TC or CC | | | | | | |

According to the method of screening multiple SNPs of the present invention, multiple SNPs associated with a specific disease or drug can be effectively selected from the entire genome of an individual.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of screening multiple single nucleotide polymorphisms (SNPs) having significance with a case group, the method comprising:
selecting one or more SNPs from nucleic acid sequences of the case group and a control group;
generating all combinable genotype patterns of multiple SNPs comprised of two or more of the selected SNPs;
determining frequencies of the genotype patterns from the case group and the control group; and
determining and choosing genotype patterns having statistical significance with the case group using the frequencies.

2. The method of claim 1, further comprising isolating substantially identical nucleic acids from a plurality of individuals of the case group and the control group before the selecting one or more SNPs from nucleic acid sequences of the case group and the control group.

3. The method of claim 1, wherein the case group has a susceptibility to a specific disease.

4. The method of claim 1, wherein the case group has no susceptibility to a specific drug or has side effects to the specific drug.

5. The method of claim 1, wherein the nucleic acid is the entire nucleic acid of individuals.

6. The method of claim 1, wherein the selecting one or more SNPs from nucleic acid sequences comprises selecting only SNPs satisfying the Hardy-Weinberg Equilibrium Law from the control group.

7. The method claim 1, wherein the multiple SNP comprises 2 to 5 SNPs.

8. The method of claim 1, wherein, when an allele of the SNP is A1/A2, the genotype patterns at the SNP site comprises the following: A1A1, A1A2, A2A2, A1A1or A1A2, and A1A2 or A2A2.

9. The method of claim 8, wherein the number of all the combinable genotype patterns of multiple SNPs comprising two or more of the selected SNPs is given by formula 2:
$${\sum }_{k = 2}^{n} {}_{n}^{}{C}{}_{k}^{} \cdot {5}^{k} , \qquad ( 2 )$$
where n=the number of SNPs.

10. The method of claim 1, further comprising creating a contingency table using the determined frequencies of the genotype patterns from the case group and the control group.

11. The method of claim 1, wherein, in the determining and choosing genotype patterns having statistical significance with the case group using the frequencies, the statistical significance is determined in consideration of a genotype pattern ratio and a genotype pattern difference.

12. The method of claim 11, wherein the statistical significance is further determined in consideration of an odds ratio, and 95% and 99% confidence intervals of the odds ratio.

13. The method of claim 12, further comprising judging that the relationship between the genotype pattern and the case group is statistically significant when the odds ratio and the lower bound of the 95% and 99% confidence intervals of the odds ratio is 1 or greater.

14. The method of claim 11, wherein the statistical significance is further determined in consideration of the p-value of Fisher's exact test.

15. The method of claim 14, further comprising judging that the relationship between the genotype pattern and the case group is statistically significant when the p-value is 0.05 or less.

16. The method of claim 14, wherein the statistical significance is further determined by correcting the p-value of Fisher' exact test.

17. The method of claim 16, wherein the correcting the p-value is performed using a multiple testing method selected from the group consisting of Bonferroni correction with discrete distributions, step-down method, step-up method, permutation method, and Bootstrap method.


SEQUENCE LISTING (48 sequences)

SEQ ID NO: 1 - DNA, 201 bp, Homo sapiens
  variation (101): n= T or G, polymorphic site
aaactgttcagtaatccttaatgtctagttctttccccaaagtacaattgcctgagtaaattatcataggtaactttgagaaggaactatgataatcatgntatataatgaggacttttctacaaggattcaggtacctcttcaatgagttctagatctagaaactgacacaagtttgggaactaggcaagaaattgtgac

SEQ ID NO: 2 - DNA, 201 bp, Homo sapiens
  variation (101): n = A or G, polymorphic site
tggagggtggcagggagctggaggagcagtgaggacttgcttgagcagtcttgacaagatgtggcaggcccacagccttcactgcctctaggccccctgantgggtcactgtggttccttcagacacaagagagaccccttattgccccagtcccactgacagactctgcctcccaggcccactggccctgcccagacatg

SEQ ID NO: 3 - DNA, 201 bp, Homo sapiens
  variation (101): n = C or A, polymorphic site
ggggtctggggagcagagaaaccaggcatctgtgagagagaaaaattaggacggacagagaggagcacagaggaggggtgcagggagagtccagggggctncattcctcctccagctatttatgtcttcaggcccaggtgcttcctacctatgggttctccaggttatccttgtgtcctctccttgtataaactccctctt

SEQ ID NO: 4 - DNA, 201 bp, Homo sapiens
  variation (101): n=T or A, polymorphic site
acctaggcagttttatctgtgtgcaaaatttagaaatgtcattcctgtggaaatgagcaaatcataaatacatcacagaaaagaagtcgctatttttttgnctttaagttgttttatagttaaattgtgtcagagagtttgccatctattttattcctaaaaaggcctggtggagaatatatgactttccttgatgtagag

SEQ ID NO: 5 - DNA, 201 bp, Homo sapiens
  variation (101): n=T or C, polymorphic site
tgcctgtgccatgaagctccgcaggggcccttccaaccctgggaatgtgttgccaacaagatcccttctgtccctgtcaggcagaggtggcagagcactcnttggcagtaagctttgtacccgaaccatttcttttttcacagtctttagataaggcagtttgagttcatttcaatagctggtacttcccgggttctgcca

SEQ ID NO: 6 - DNA, 201 bp, Homo sapiens
  variation (101): n=A or T, polymorphic site
agcgtagtagcaaactgctggcccacagcctgctatgaagtaggagttcattaccttcttcgctccaggtcttgacatggtccaaagacttgtcttttgangcagccctgttgtatcctcttgagttgtcatgacattgtctgctggtcttccagtggcaaaatatcctagactttcagagctgaaaaaaaaaggtacttt

SEQ ID NO: 7 - DNA, 201 bp, Homo sapiens
  variation (101): n= A or G, polymorphic site
ggaagcctcctctgcccttgcctctcttggaactcgaggtccaccctgacaaagccacactgggtcccagccgcagtgtctctcctggcccagggagcacntacctggatcctcctccttcaccgtaaagttgtagctggactgattgaagatgggcaggttatcattgatgtcctgcagtcagagacaggtctgagtccc

SEQ ID NO: 8 - DNA, 201 bp, Homo sapiens
  variation (101): n=T or C, polymorphic site
aggcaggctctgaactcaggcacccccagagctggatcctgttcgctcctcttaatggtcatgcgtgggatcttatttaacctctttaagccctggcttcncatctgcaaattggcaatgataatggtgcaagcctcatggagctgtgagaattaaatgaagcatatgtgtgtaaaagcagttggcacagtacttggcata

SEQ ID NO: 9 - DNA, 201 bp, Homo sapiens
  variation (101): n=A or G, polymorphic site
atggtttttaactcttttctgaaaacattttcagatgacattcctgaaagctcactcttctcaccaatgccatcagaggaaaaggctgcttcccctcccanacctctgctttcaaatgcctcggcaactccagttggcagaaggggccgtctggccaatcttgctgcaactatttgctcctgggaagatgatgtaaatcac

SEQ ID NO: 10 - DNA, 201 bp, Homo sapiens
  variation (101): n=A or G, polymorphic site
tttggagattagctaataggtacagtctcctttcccatcaaaccttttactggaaccacaatgaaatagtacatttgattgattcaaacttcagctttttntttcttactatctttcccttgtaaattaaactacccaaaattttaatgaccaaaatagaaattatcagcagaaagatagtaacaaccaactgaaggaatt

SEQ ID NO: 11 - DNA, 201 bp, Homo sapiens
  variation (101): n=A or T, polymorphic site
acccactctacagagatctgagctctgcagggcccctccgtgataaacagaatcagtcagagggggacttaactcagtggctaaggacttttctccaagtntggaggtcctgataaccctgacttcattaaccctttattgccctagccagtggggtgggagggagagattgtttcctgcccttttttcgttgttatatca

SEQ ID NO: 12 - DNA, 201 bp, Homo sapiens
  variation (101): n=A or T, polymorphic site
attttttcttctcttagtaccacttccttcagtcaaataatttccaagtcctgtcagtgacatgttgaaaatgacaattcttgtcatctgagaagcacttnttgtcaatgcgtaacttcaaagctcatcatctctcatttgaattattgtgataacataccaatcctgtttcttttttcatattcacatcttcttaagtta

SEQ ID NO: 13 - DNA, 30 bp, Artificial Sequence
  primer
acgttggatgcataggtaactttgagaagg

SEQ ID NO: 14 - DNA, 30 bp, Artificial Sequence
  primer
acgttggatgaggtacctgaatccttgtag

SEQ ID NO: 15 - DNA, 23 bp, Artificial Sequence
  primer
gagaaggaactatgataatcatg

SEQ ID NO: 16 - DNA, 30 bp, Artificial Sequence
  primer
acgttggatgagcagtcttgacaagatgtg

SEQ ID NO: 17 - DNA, 30 bp, Artificial Sequence
  primer
acgttggatgtgtgtctgaaggaaccacag

SEQ ID NO: 18 - DNA, 19 bp, Artificial Sequence
  primer
ctgcctctaggccccctga

SEQ ID NO: 19 - DNA, 30 bp, Artificial Sequence
  primer
acgttggatgggcctgaagacataaatagc

SEQ ID NO: 20 - DNA, 30 bp, Artificial Sequence
  primer
acgttggatgaaattaggacggacagagag

SEQ ID NO: 21 - DNA, 21 bp, Artificial Sequence
  primer
taaatagctggaggaggaatg

SEQ ID NO: 22 - DNA, 30 bp, Artificial Sequence
  primer
acgttggatgcatcacagaaaagaagtcgc

SEQ ID NO: 23 - DNA, 30 bp, Artificial Sequence
  primer
acgttggatgtagatggcaaactctctgac

SEQ ID NO: 24 - DNA, 23 bp, Artificial Sequence
  primer
gaaaagaagtcgctatttttttg

SEQ ID NO: 25 - DNA, 30 bp, Artificial Sequence
  primer
acgttggatgggttcgggtacaaagcttac

SEQ ID NO: 26 - DNA, 30 bp, Artificial Sequence
  primer
acgttggatgatgtgttgccaacaagatcc

SEQ ID NO: 27 - DNA, 21 bp, Artificial Sequence
  primer
gggtacaaagcttactgccaa

SEQ ID NO: 28 - DNA, 30 bp, Artificial Sequence
  primer
acgttggatggaccagcagacaatgtcatg

SEQ ID NO: 29 - DNA, 30 bp, Artificial Sequence
  primer
acgttggatgggtcttgacatggtccaaag

SEQ ID NO: 30 - DNA, 19 bp, Artificial Sequence
  primer
gaggatacaacagggctgc

SEQ ID NO: 31 - DNA, 29 bp, Artificial Sequence
  primer
acgttggatgtgacaaagccacactgggt

SEQ ID NO: 32 - DNA, 30 bp, Artificial Sequence
  primer
acgttggatgtacggtgaaggaggaggatc

SEQ ID NO: 33 - DNA, 18 bp, Artificial Sequence
  primer
tcctggcccagggagcac

SEQ ID NO: 34 - DNA, 30 bp, Artificial Sequence
  primer
acgttggatgcttgcaccattatcattgcc

SEQ ID NO: 35 - DNA, 30 bp, Artificial Sequence
  primer
acgttggatgtcttaatggtcatgcgtggg

SEQ ID NO: 36 - DNA, 17 bp, Artificial Sequence
  primer
ttgccaatttgcagatg

SEQ ID NO: 37 - DNA, 30 bp, Artificial Sequence
  primer
acgttggatggctcactcttctcaccaatg

SEQ ID NO: 38 - DNA, 30 bp, Artificial Sequence
  primer
acgttggatggttgccgaggcatttgaaag

SEQ ID NO: 39 - DNA, 17 bp, Artificial Sequence
  primer
ggctgcttcccctccca

SEQ ID NO: 40 - DNA, 31 bp, Artificial Sequence
  primer
acgttggatggggtagtttaatttacaaggg

SEQ ID NO: 41 - DNA, 30 bp, Artificial Sequence
  primer
acgttggatgctggaaccacaatgaaatag

SEQ ID NO: 42 - DNA, 23 bp, Artificial Sequence
  primer
tacaagggaaagatagtaagaaa

SEQ ID NO: 43 - DNA, 30 bp, Artificial Sequence
  primer
acgttggatgctggctagggcaataaaggg

SEQ ID NO: 44 - DNA, 30 bp, Artificial Sequence
  primer
acgttggatgtcagtggctaaggacttttc

SEQ ID NO: 45 - DNA, 20 bp, Artificial Sequence
  primer
cagggttatcaggacctcca

SEQ ID NO: 46 - DNA, 30 bp, Artificial Sequence
  primer
acgttggatggatgagctttgaagttacgc

SEQ ID NO: 47 - DNA, 30 bp, Artificial Sequence
  primer
acgttggatggtcctgtcagtgacatgttg

SEQ ID NO: 48 - DNA, 18 bp, Artificial Sequence
  primer
gaagttacgcattgacaa